(19) United States
(12) Patent Application Publication
(10) Pub. No.: US 2007/0100408 A1
(43) Pub. Date: May 3, 2007

(54) Electrical stimulation lead with proximal common electrode

(75) Inventor: Martin T. Gerber, Maple Grove, MN (US)

Correspondence Address: SHUMAKER & SIEFFERT, P. A., 1625 RADIO DRIVE, SUITE 300, WOODBURY, MN, 55125, US

(73) Assignee: Medtronic, Inc., Minneapolis, MN (US)

(21) Appl. No.: 11/261,406

(22) Filed: Oct. 28, 2005

(51) Int. Cl. A61N 1/00

(52) U.S. Cl. 607/117

(57) ABSTRACT

An implantable medical lead for stimulation of target sites, including nerves, organs, or tissue comprises a lead body having a distal electrode lead segment and a proximal connector lead segment. The electrode lead segment has at its distal end an electrode array including at least one stimulation electrode. The connector lead segment includes at least one common electrode. The proximal end of the connector lead segment may be coupled to a pulse generator, other stimulation device, or additional intermediate wiring.

### FIELD OF THE INVENTION

This invention relates generally to implantable stimulation leads for stimulation of target sites within a patient, such as nerves, organs or other tissue.

### BACKGROUND

Millions of people of all ages suffer from urinary urge incontinence, nonobstructive urinary retention, or significant symptoms of urgency-frequency, as well as other types of pelvic floor disorders including pelvic pain, bowl dysfunction (constipation, diarrhea) and erectile dysfunction. Individuals with these conditions often face debilitating challenges in their everyday lives. They can be preoccupied with constant trips to the bathroom, fear of leaking episodes, and sleepless nights. Many sufferers become so anxious about their conditions that they become isolated and depressed.

Although many people suffer from bladder control problems or other types of pelvic floor disorders, there are limited treatment options to help relieve symptoms. More conservative treatments currently available include behavioral techniques (healthy lifestyle habits, diet modification, biofeedback, bladder retraining, pelvic muscle exercises) and medications (anticholinergics, antispasmodics, antimuscarinics, or tricyclic antidepressants). However, many of the pharmaceuticals used to treat these disorders do not adequately resolve the issue and can cause unwanted side effects. Surgical procedures currently available such as bladder augmentation, bladder denervation, or bladder removal may have a low success rate and are irreversible. When available treatments are not effective or indicated, patients manage bladder control problems through the use of external collection devices such as catheters or absorbent pads or undergarments.

The organs involved in bladder, bowel, and sexual function receive much of their control via the second, third, and fourth sacral nerves, commonly referred to as S2, S3 and S4 respectively. Electrical stimulation of these various nerves has been found to offer some control over these functions. Thus, for example, medical leads having discrete electrode contacts have been implanted on and near the sacral nerves of the human body to provide partial control for bladder incontinence. Unlike other surgical procedures, sacral nerve stimulation using an implantable pulse generator is fully reversible simply by turning off the pulse generator or removing the implanted device.

### SUMMARY

In general, the invention is directed to an electrical stimulation lead for stimulation of target site within the body of patient, including but not limited to nerves, organs, or other tissue. The electrical stimulation lead includes an electrode lead segment having at its distal end an electrode array including at least one stimulation electrode. The electrical stimulation lead also includes a connector lead segment that has at its proximal end at least one common electrode.

In one embodiment, the invention is directed to an electrical stimulation lead comprising an electrode lead segment having a distal electrode array adapted to deliver neurostimulation pulses to a target site and a connector lead segment having a proximal common electrode adapted to serve as a return electrode. The electrode array includes at least one stimulation electrode. In some embodiments, the electric field produced between the electrode array and the common electrode simulates a monopolar stimulation field applied between an electrode array and an implanted neurostimulator housing.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIG. 1 is a plan view of an example embodiment of an electrical stimulation lead having a proximal connector segment and a distal electrode segment.

FIG. 2 is a schematic illustration of an electrical stimulation lead implanted near the sacral nerve of a patient.

### DETAILED DESCRIPTION

The invention provides an implantable electrical stimulation lead for stimulation of target sites with the body of patient such as nerves, organs, or other tissue. The invention comprises a neurostimulation lead having an electrode segment that includes a distally positioned stimulating electrode array. The lead also includes a connector segment that includes at least one proximally positioned common electrode. The common electrode provides a return path for the electrical stimulation pulses delivered by the stimulating electrodes. The electrical stimulation lead may be used, for example, for control of incontinence or other pelvic floor disorders by stimulation of targeted nerves or tissue, such as the sacral nerves.

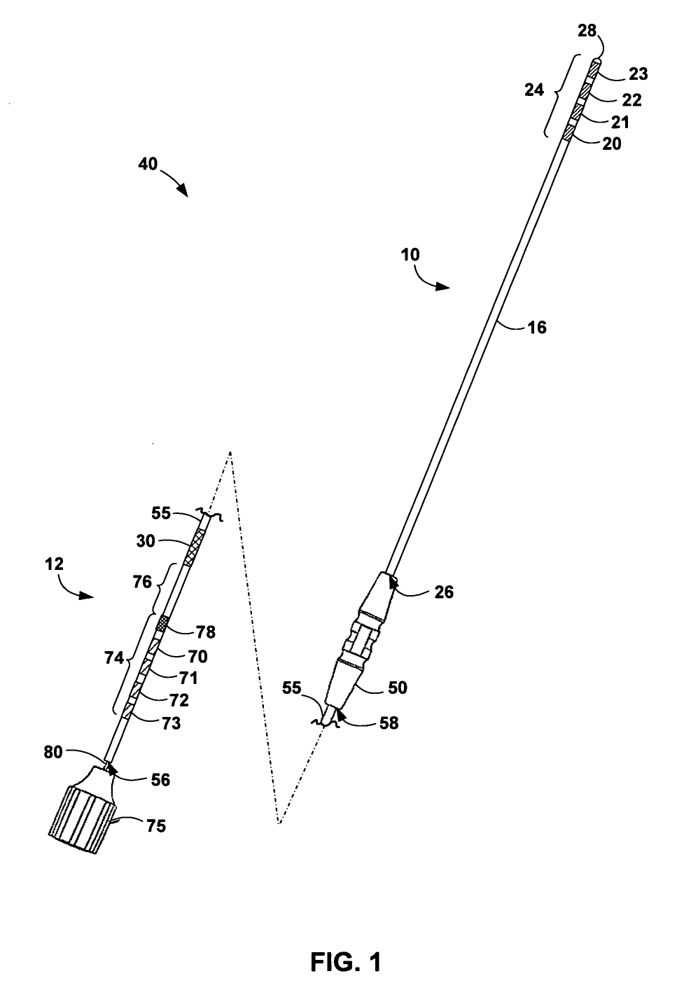

FIG. 1 shows an example embodiment of an electrical stimulation lead 40. Lead 40 includes a proximal connector segment 12 and distal electrode segment 10. A connector 50 connects the distal electrode segment 10 and the proximal connector segment 12. In use, lead 40 is implanted in the body of a patient with electrode segment 10 positioned proximate the target nerves or tissue to be stimulated.

Electrode segment 10 includes at its distal end 28 an electrode array 24 having at least one stimulation electrode. In the example embodiment shown in FIG. 1, electrode array 24 is a quadripolar electrode array comprised of four annularly-shaped ring electrodes 20, 21, 22, and 23. Stimulating electrodes 20, 21, 22, and 23 are coupled through separate conductors (not shown) extending through an interior lumen within lead bodies 16 and 55 to corresponding connector elements 70, 71, 72, and 73 of a connector element array 74 positioned at the proximal end 56 of connector segment 12.

Connector element array 74 may be coupled to an external or implantable neurostimulation pulse generator, also known as a neurostimulator, additional intermediate wiring, or other stimulation device. An example of such an implantable neurostimulation pulse generator is the Medtronic InterStim Neurostimulator. Stimulation pulses produced by the neurostimulator coupled to the connector elements 70, 71, 72, and/or 73 at the proximal end 35 of the lead body 55 are conducted through the aforementioned conductors extending from connector element array 74 to the desired stimulation electrodes 20, 21, 22, and/or 23 within electrode array 24.

A common electrode 30, separated from the distal end of connector element array 74 by a distance 76, functions as an indifferent electrode and provides a return path for the neurostimulation pulses delivered by electrode array 24. Connector element 78 of connector array 74 provides for connection of common electrode 30 back to the neurostimulation pulse generator.

A stylet includes an elongated stylet wire 80 that can be inserted into or retracted from the interior lumen of the lead bodies 55, 16 by manipulation of a stylet handle 75 attached at the proximal end of the stylet wire 80. Stylet 80 may be made of solid wire such as tungsten or stainless steel. Stylet 80 stiffens lead bodies 55, 16 to provide support to lead 40 during implantation. After implantation is complete, stylet 80 may be removed.

Electrical stimulation lead 40 may range between about 20 centimeters and 60 centimeters in total length, depending on the location of the site to be stimulated and the distance of the neurostimulator from such site. However, other lead lengths such as those less than 20 centimeters or greater than 60 centimeters are also contemplated in the present invention. Lead bodies 16 and 55 may be less than about 5 mm in diameter, and in some embodiments are less than about 1.5 mm in diameter. Lead bodies 16 and 55 may be formed of polyurethane, silicone, or of any other appropriate biocompatible material known in the art. The internal electrical conductors connecting the electrode array 24 with the connector array 74 for supplying electrical current to the electrodes may be formed of coiled, braided or stranded wires comprising, for example, an MP35N platinum-iridium alloy or other suitable material known in the art.

Electrical stimulation pulses generated by a neurostimulator may be applied to the targeted nerves or tissue through one or more of the electrodes 20, 21, 22, and/or 23 in distal electrode array 24. For example, the stimulation pulses may be delivered between a selected active one of the stimulation electrodes 24 and the common electrode 30, which provides a remote, indifferent or return electrode. In this case, efficacy of stimulation between each of the electrodes 20, 21, 22, or 23 in distal electrode array 24, either alone or in combination with some or all of the other electrodes in distal electrode array 24, and common electrode 30 may be tested, and the most efficacious electrode or combination of electrodes may be selected for use. In this manner, one electrode, some combination of electrodes, or all of the electrodes in electrode array 24 may be used for stimulation depending upon the size of stimulating field to be applied and the desired physiological response.

The electrical pulse stimulation parameters may also be adjusted to optimize the therapy delivered to the patient. Such adjustment may entail one or more of adjusting the number or configuration of electrodes or leads used to stimulate the selected location, pulse amplitude, pulse frequency, pulse width, pulse morphology (square wave, triangle wave, sinusoid, tri-phasic pulse, etc.), times of day or night when pulses are delivered, pulse cycling times, the positioning of the lead or leads, and the enablement or disablement of “soft start” or ramp functions respecting the stimulation regime to be provided.

Electrodes 20, 21, 22 and 23 may be ring electrodes, coiled electrodes, mesh electrodes, electrodes formed from portions of wire, barbs, hooks, spherically-shaped members, helically-shaped members, or may assume any of a number of different structural configurations well known in the art. It shall be understood, therefore, that the present invention is not limited with respect to the particular size, shape, or number of electrodes in the electrode array 24 or the common electrode 30. Examples of various electrode types which may be used with the electrical lead of the present invention are described in U.S. Pat. No. 6,055,456 to Gerber et al., dated Apr. 25, 2000, United States Patent Application Publication U.S. 2001/0025192 to Gerber et al., published Sep. 27, 2001, and United States Patent Application Publication U.S. 2005/0113877 to Spinelli et al., published May 26, 2005, all of which are incorporated herein by reference in their entireties. Thus, although FIG. 1 shows four electrodes located at the distal end of lead 40, other electrode configurations are possible and contemplated in the present invention.

In the case of an electrode segment 24 having four ring electrodes such as that shown in FIG. 1, inter-electrode distances on electrode segment 24 may be anywhere between about 0.5 mm and about 5 mm. The surface areas of electrodes 20, 21, 22 and 23 may be anywhere between about 1.0 sq. mm and about 50 sq. mm. Electrodes 20, 21, 22 and 23 may have a length anywhere between about 0.25 mm and about 20 mm. Stimulation electrodes 20, 21, 22 and 23 may be formed of platinum, stainless steel, gold, or any other biocompatible metals or metal alloys known to those of skill in the art.

The size, shape, and number of electrodes within the electrode array 24, the size, shape, and number of common electrodes 30, and the distance between the common electrode 30 and the electrode array 24 are chosen such that the stimulating electric field is sufficiently broad to produce the desired physiologic response, as well as minimizing tissue stimulation at the common electrode site. To that end, in some embodiments, the size of common electrode 30 is chosen to be similar in size (i.e., surface area) to the smallest one of the stimulation electrode in electrode array 24. In other embodiments, the surface area of common electrode 30 may be at least 1.5 times greater than the largest one of the stimulation electrodes in electrode array 24. In still other embodiments, the surface area of common electrode 30 may be anywhere between about 2 and 10 times greater than the largest one of the stimulation electrodes in electrode array 24. Common electrode 30 may be formed of platinum, stainless steel, gold, or any other biocompatible metals or metal alloys known to those of skill in the art.

The electric field produced between the electrode array and the common electrode may simulate a monopolar stimulation field such as that applied between a stimulation electrode array and an implanted neurostimulator housing used as a return electrode. In some embodiments, the size and shape of common electrode 30, and the distance between common electrode 30 and electrode array 24 is selected such that common electrode 30 approximates or approaches an infinitely large electrode relative to the largest one of the distal stimulation electrodes. In this sense, common electrode may be selected to have a size (i.e., surface area) at least twice as large as the largest one of the stimulating electrodes in electrode array.

The distance between the distal end of common electrode 30 and the proximal end of electrode array 24 may be anywhere between about 3 centimeters to about 50 centimeters. The distance between the distal end of common electrode 30 and the proximal end of electrode array 24 may depend upon, among other things, the total length of lead 40, the size of the connector element 74, the size of common electrode 30, the size of electrode array 24, and the size of the neurostimulation field required to produce the desired physiologic response.

The distance 76 between the distal end of connector element 74 and the proximal end of common electrode 30 may be anywhere between about 1 centimeter to about 20 centimeters. This distance 76 may depend upon, among other things, the practical distance which would allow for connection of lead 40 to the neurostimulator via connector element 74, the overall length of lead 40, and the desired distance between common electrode 30 and electrode array 24. In some embodiments, such as the embodiment shown in FIG. 1, common electrode 30 is positioned along the lead body relatively closer to connector element 74 than to electrode array 24.

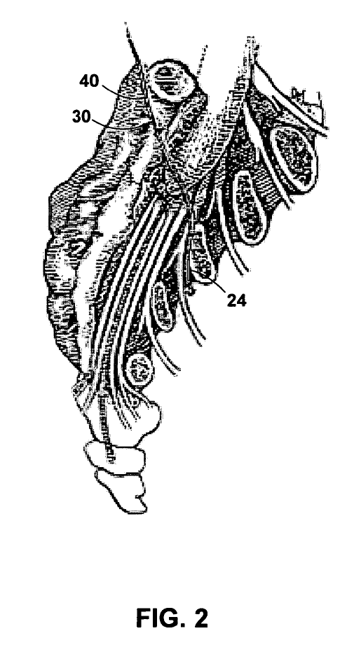

FIG. 2 shows an overall schematic of the sacral nerve area with an electrical stimulation lead 40 implanted near a sacral nerve for stimulation. Lead 10 is inserted by first making an incision appropriate to the size of the patient and then splitting the paraspinal muscle fibers to expose the sacral foramen. The physician then locates the desired position and inserts lead 10 into the foramen and may anchor lead 10 in place. Lead 10 is placed close enough to the nerve bundle that the electrical stimulation results in the desired physiological responses. The desired effect varies depending on which pelvic floor disorder is being treated and/or which nerve or nerves are being stimulated. Lead 10 is implanted in close proximity of the nerve to result in the most efficient transfer of electrical energy.

For control of incontinence, for example, the physician may implant lead 10 near the S3 sacral nerves. Lead 10 may, however, be inserted near any of the sacral nerves including the S1, S2, S3, or S4, sacral nerves depending on the necessary or desired physiologic response. Lead 10 may also be inserted for stimulation of any nerves along the spinal column. Lead 10 may be used to stimulate multiple nerves or multiple sides of a single nerve bundle. In addition, lead 10 may also be used as an intramuscular lead. This may be useful in muscle stimulation such as dynamic graciloplasty.

In use, the electrode segment 10, and particularly electrode array 24, of lead 40 extends to a target site such as a position near a desired nerve or nerve portion, organ, or tissue and may be held in such position by a lead anchor (not shown). The lead anchor may assume any of a number of different structural configurations such one or more suture sleeves, tines, barbs, hooks, a helical screw, tissue in-growth mechanisms, adhesive or glue. Examples of suitable lead anchors are described in United States Patent Application Publication U.S. 2005/0113877 A1 to Spinelli et al., published May 26, 2005, which is incorporated herein by reference in its entirety. Other lead anchors known in the art may also be used.

It is contemplated that drugs may be delivered to specific sites within a body of a patient using well known drug pump devices, either external or implantable, in combination with providing electrical stimulation to targeted sites such as nerves, organs, or tissue as described above. In this case, the drug pump may be incorporated into the same housing as the neurostimulator, or may be separate therefrom in its own hermetically sealed housing. The drug catheter attached to the drug pump through which the drug is delivered to the specific site may also incorporated into lead 40, or may be separate therefrom. Drugs or therapeutic agents that may be delivered in accordance with this technique include, but are not limited to, antiobioticsd, pain relief agents such as Demerol and morphine, radioactive or radio-therapeutic substances or agents for killing or neutralizing cancer cells, genetic growth factors for encouraging the growth of healthy tissues, drugs for facilitating or encouraging sexual function, and other drugs capable of being delivered via a drug pump as are known in the art.

One advantage provided by the electrical stimulation lead 40 with proximal common electrode 30 described herein is that a test stimulation allows patient assessment prior to implant of a neurostimulator. This test stimulation procedure assesses the effect of electrical stimulation therapy for each patient prior to consideration of a surgical implant procedure. For example, the test stimulation demonstrates the effect of sacral nerve stimulation on patient symptoms over a 3-5 day trial period. The test stimulation also allows the patient to experience the sensation of stimulation during various everyday activities. As a result, the test stimulation provided by the electrical stimulation lead 40 helps the physician and the patient make an informed choice about electrical stimulation therapy as a long-term therapy option, and allows them to evaluate the therapy as an option for the patient before the commitment of an implant. Moreover, it also provides patients with realistic expectations about the results of electrical stimulation therapy. If the test stimulation is successful, the patient may go on to the implantation of the electrical stimulation lead and implantable neurostimulator.

For example, a staged lead implant procedure may be followed in which, during the percutaneous test stimulation, an electrode lead segment 10 is implanted within the patient and an externalized extension, such as connector segment 12, is connected to an external neurostimulator. After the test simulation period, if the patient meets the criteria for long-term therapy, the implantation of the implantable neurostimulator and extension can proceed. If the benefit to the patient is not demonstrated, the lead may be either repositioned for another test or removed.

Advantages of the electrical stimulation lead with proximal common electrode of the type described herein include increased accuracy of the test stimulation procedure. For example, because the stimulation electrode array 24 and the common (return) electrode 30 are both implanted within the patient, a more accurate gauge of the effectiveness of the neurostimulation can be achieved. Because the entirety of the stimulation delivery mechanism (namely both the source and return electrodes) are implanted in the desired location within the patient, the resulting stimulation applied to the patient during the test stimulation is precisely the same as that which would be applied once the entire neurostimulation device is implanted. This increase in accuracy of the test stimulation may be of particular help to patients and their physicians when evaluating the effectiveness of electrical stimulation therapy.

In addition, both time and expense of implantation may be reduced and accuracy may be increased because no ground pad to simulate the return path to an implanted housing or “can” of a neurostimulator is required.

Another advantage provided by the electrical stimulation lead of the present invention is more rapid placement of the electrodes during the implantation procedure. The neurostimulation provided by electrode array 24 and common electrode 30 may create a broader, stronger electric field, which allows the lead to be placed in a less precise manner while still providing adequate electrical stimulation to the targeted nerves or other desired areas. The ability to implant the lead with less specificity as to the location near the targeted nerves may therefore reduce the time required for implantation as well as increase the likelihood that the therapy will be successful.

Other issues mitigated through use of a common electrode of type described herein may include improvement of neurostimulator defibrillation protection, improvement of EMI protection/performance and a decrease in cost of manufacture.

Various embodiments of the invention have been described. These and other embodiments are within the scope of the following claims.

1. An electrical stimulation lead comprising:
an electrode segment having a distal electrode array adapted to deliver neurostimulation pulses to a target site; and
a connector segment having a proximal common electrode adapted to serve as a return electrode for electrodes of the electrode array.

2. The lead of claim 1, wherein the connector segment is connected to a proximal end of the electrode segment and receives the neurostimulation pulses from a neurostimulator.

3. The lead of claim 2, wherein the neurostimulator is one of an external neurostimulator or an implantable neurostimulator.

4. The lead of claim 1, wherein the electrode array includes at least one stimulation electrode.

5. The lead of claim 1, wherein the electrode array includes four ring electrodes.

6. The lead of claim 1, wherein the electrode array includes at least two ring electrodes, and wherein the common electrode includes at least one ring electrode having a larger surface area than a largest one of the ring electrodes in the electrode array.

7. The lead of claim 1, wherein the electrode array and the common electrode are separated by a distance of at least 3 centimeters.

8. The lead of claim 1, wherein the electrode array and the common electrode are separated by a distance of at least 5 centimeters.

9. The lead of claim 1, wherein the electric field produced between the electrode array and the common electrode simulates a monopolar stimulation field applied between an electrode array and an implanted neurostimulator housing.

10. The lead of claim 1, wherein the electrode array includes at least one of a wire coil electrode, a mesh electrode, a ring electrode, an electrode formed from portions of wire, barbs, hooks, spherically-shaped members, and helically-shaped members.

11. The lead of claim 1, wherein the electrode array includes at least one stimulation electrode, and wherein the common electrode has a size at least as large as the at least one stimulation electrode.

12. The lead of claim 1, wherein the electrode array includes at least two stimulation electrodes, and wherein the common electrode includes at least one return electrode having a larger surface area than a largest one of the stimulation electrodes in the electrode array.

13. The lead of claim 12, wherein the common electrode has a surface area at least two times as great as a surface area of the largest one of the stimulation electrodes in the electrode array.

14. The lead of claim 1, wherein the electrode array and the common electrode provide neurostimulation of at least one sacral nerve.

15. The lead of claim 1, wherein the electrode array is adapted for implantation to deliver neurostimulation pulses to at least one sacral nerve.

16. The lead of claim 1, wherein the common electrode is relatively closer to the connector element than to the electrode array.

17. The lead of claim 16, wherein a distance between the connector element and the common electrode is at least 1 centimeter, and wherein a distance between the common electrode and the electrode array is at least 5 centimeters.

18. An electrical stimulation lead, comprising:
a lead body comprised of an electrode segment and a connector segment, the electrode segment having a distal electrode array adapted to deliver neurostimulation pulses to a target site;
the connector segment including a connector element adapted to receive the neurostimulation pulses from a neurostimulator;
the connector segment further including a proximal common electrode adapted to serve as a return electrode for electrodes of the electrode array;
wherein the common electrode is positioned along the lead body relatively closer to the connector segment than to the electrode array.

19. The lead of claim 18, wherein the lead is configured for implantation within the body of a patient to stimulate a target site and wherein the common electrode serves as a return electrode during a test stimulation procedure.

20. The lead of claim 19, wherein the neurostimulation pulses are received from an external neurostimulator during the test stimulation procedure.
